(19) United States
(12) Patent Application Publication
(10) Pub. No.: US 2025/0195743 A1
(43) Pub. Date: Jun. 19, 2025

(54) Multifunctional Rotary Rinser

(71) Applicant: Sheng Huang, Ziyang (CN)

(72) Inventor: Sheng Huang, Ziyang (CN)

(21) Appl. No.: 18/970,426

(22) Filed: Dec. 5, 2024

(30) Foreign Application Priority Data
Dec. 13, 2023 (CN) 2023234079800

(51) Int. Cl. A61M 3/02

(52) U.S. Cl. CPC A61M 3/02

(57) ABSTRACT

This application is introduced herein related to the multifunctional rotary rinser, including handle and swivel; one end of the handle is provided with water inlet and the other end rotationally connected to the swivel, and water channel runs through both swivel ends; the handle is also installed with the motor; this invention herein installs pump in the handle to connect the water inlet and water channel, and when using, no longer need to rely on the role of the water heater shower, so that the outdoor and indoor use become very convenient; and at the same time, according to the need, the swivel replacement could realize the medical anus rinsing and daily cleaning vagina rinsing; the functional diversification improves the practical value; the motor can drive the swivel to rotate, so as to achieve a better rinsing effect and the purpose of rinsing without dead corners.

### TECHNICAL FIELD

This application is introduced herein related to the rinsers, in particular, to the multifunctional rotary rinser.

### BACKGROUND OF THE PRESENT INVENTION

For a long time, due to technical and manufacturing constraints, the gynecological rinsers available on the market are generally low-tech squeeze-type disposable rinsers. Although this type of rinser is cheaper and simpler to operate, it has fundamental shortcomings and poses hidden dangers to women's health. There are several significant drawbacks to the traditional female rinser:
1. The process is rough, as burrs are formed due to its one-time blow molding; container and washing head are linked, once the container is squeezed, the washing head would greatly shifted, thus it is easy to scratch the vagina and cervical epithelial tissue, aggravating the condition.
2. The washing rod is too long, so the cervical head will be injured unintentionally; and the washing head is too sharp, so it is easy to cut the vagina.
3. The volume is too small, usually in 50 milliliters or less.
4. The sewage backflow; the water outlet and air inlet of the traditional rinsers are designed on the top, so when the squeeze release, the sewage backflow, and the backflow contaminates the rinser.
5. Some of the washing head is cone-shaped with pointed top and large bottom. When rinsing, it blocks the vaginal orifice, easily forming a greater pressure of vaginal than the uterus, and prompting pathogenic microorganisms up to the uterus and fallopian tubes.

In response to the above problems, a Chinese patent with publication number CN108404244A discloses a gynecological rinser, which solves the above problems. However, it can only be used indoors in conjunction with the water heater shower hose. The water power is provided by the water heater shower, which is too restrictive, and it is troublesome or even impossible to use in a place where there is no water heater shower, and also limits its usage scenarios, making it single function and limiting its value.

### Summary of Present Invention

The purpose of the present invention herein is to provide the multifunctional rotary rinser in order to solve the above mentioned problems raised in the background.

In order to realize the above object, the present invention herein adopts the following technical solution:

Multifunctional rotary rinser, characterized in that it comprises: handle and swivel; one end of the handle is provided with the water inlet; the swivel is rotationally connected to the other end of the handle; swivel is provided with water channel running through the ends of the swivel; pump is installed in the handle; the inlet of the pump is connected to the water inlet; the outlet of the pump is connected to the water channel through the pipe; the said handle is also installed with motor driving the swivel.

As a preferred technical solution in the present invention herein, the said handle includes water inlet end cap, handle sleeve and water outlet end cap, which are connected sequentially from one end to the other end of the handle; the water inlet of the handle is set on the water inlet end cap; the swivel is rotationally connected to the water outlet cap; the water outlet end cap is provided with the handle outlet pipe connected to the water channel; the outlet of the pump is connected to the handle outlet pipe; the electric motor is installed on the water outlet end cap.

As a preferred technical solution in the present invention herein, motor shaft of the said motor runs through the water outlet end cap and is coaxially connected with the driving gear; driven gear mounted in the swivel; driven gear is meshed with driving gear; the said driven gear is fixed inside the swivel.

As a preferred technical solution in the present invention herein, the said handle outlet pipe comprises inlet channel, adapter channel and outlet channel connected sequentially along the water flow direction; the end of the swivel close to the handle s socketed outside the water outlet end cap; the middle part of the swivel is rotationally connected to the water outlet channel through bearing.

As a preferred technical solution in the present invention herein, battery for supplying power to pump and motor is installed inside the said handle; charging port for charging battery is provided on handle; the said handle is also equipped with the processor; the processor is electrically connected to pump, motor, and battery.

As a preferred technical solution in the present invention herein, the said handle is further provided with control button and power display screen, both of which are electrically connected to the processor.

As a preferred technical solution in the present invention herein, the said swivel is covered with silicone case; water outlet is provided on the silicone case.

As a preferred technical solution in the present invention herein, protective cover is provided outside the said swivel.

Beneficial effects: this invention herein installs pump in the handle, using the pump to connect the water inlet and water channel, and then in the use of this multifunctional rotary rinser, no longer need to rely on the role of the water heater shower, so that the outdoor and indoor use become very convenient; and at the same time, according to the need, the swivel replacement could realize the medical anus rinsing and daily cleaning vagina rinsing; the functional diversification improves the practical value; the swivel function ensures that the product will wash away the dirt inside the folds of the female vagina when flushing; when carrying out medical anal rinsing, the motor can drive the swivel to rotate, and making the water out of the channel to form a rotating water spray effect, so as to achieve a better rinsing effect and the purpose of rinsing without dead corners.

### BRIEF DESCRIPTION OF THE DRAWINGS

FIG. 1 illustrates the schematic diagram of the structure of the invention herein;

FIG. 2 illustrates the schematic diagram of the structure of the invention herein when the protective cover is not installed;

FIG. 3 illustrates the schematic diagram of the structure of the invention herein when the silicone sleeve is not installed in the situation shown in FIG. 2;

FIG. 4 illustrates the schematic diagram of the structure of the invention herein when the swivel is not installed in the situation shown in FIG. 3;

FIG. 5 illustrates the schematic diagram of the structure of the invention herein when the handle sleeve is not installed in the situation shown in FIG. 4;

FIG. 6 illustrates the schematic diagram of the structure of the fit of driving gear, swivel, and bearing of the invention herein;

FIG. 7 illustrates the sectional schematic diagram of the structure of the invention herein;

The appended drawings: 1-handle; 101-water inlet end cap; 102-handle sleeve; 103-water outlet end cap; 104-handle outlet pipe; 2-swivel; 202-driven gear; 3-pump; 4-motor; 5-driving gear; 6-battery; 7-silicon case; 8-protective cover; 9-bearing.

### DETAILED DESCRIPTION OF THE EMBODIMENT'S

In order to more clearly illustrate the technical solutions in the embodiments or current technology of the present invention herein, the present invention will be briefly introduced in the following in conjunction with the appended drawings and the description of the embodiments or current technology; it is obvious that the following description of the structure of the appended drawings is only some of the embodiments of the present invention herein, and that other drawings may be obtained by a person of ordinary skill in the field on the basis of the drawings, without any creative labor; it should be noted herein that the structure of this invention is only an example of some embodiments. It should be noted that the description of these embodiments is intended to assist in the understanding of the present invention herein, but does not constitute a limitation of the present invention herein.

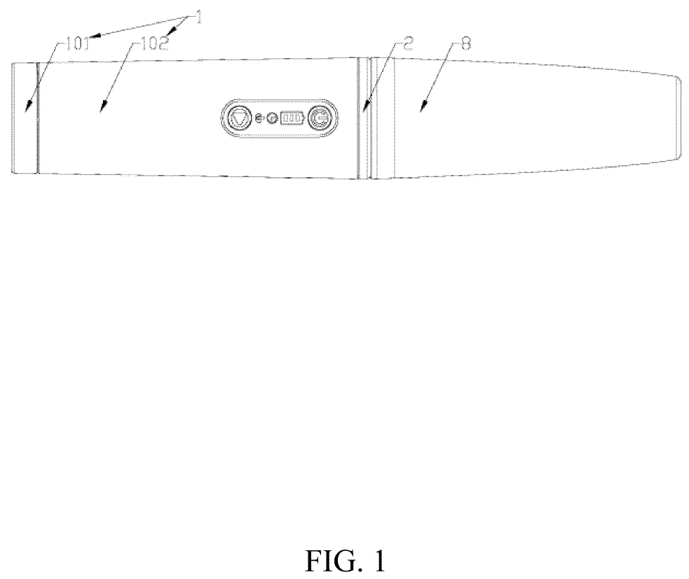

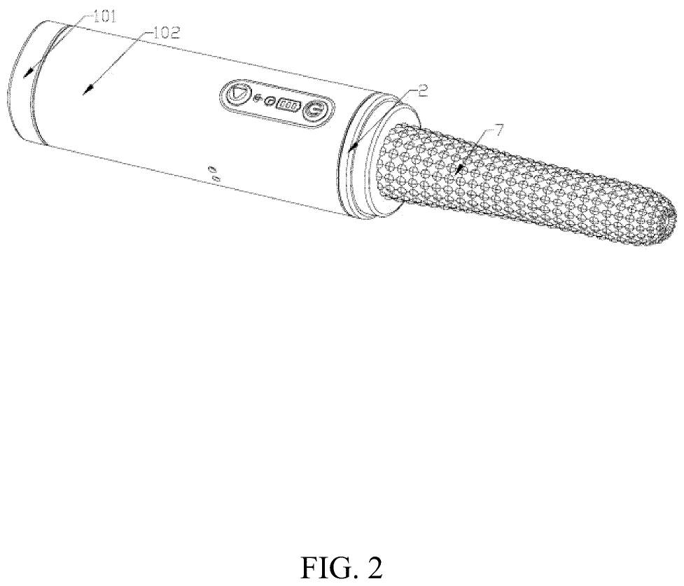

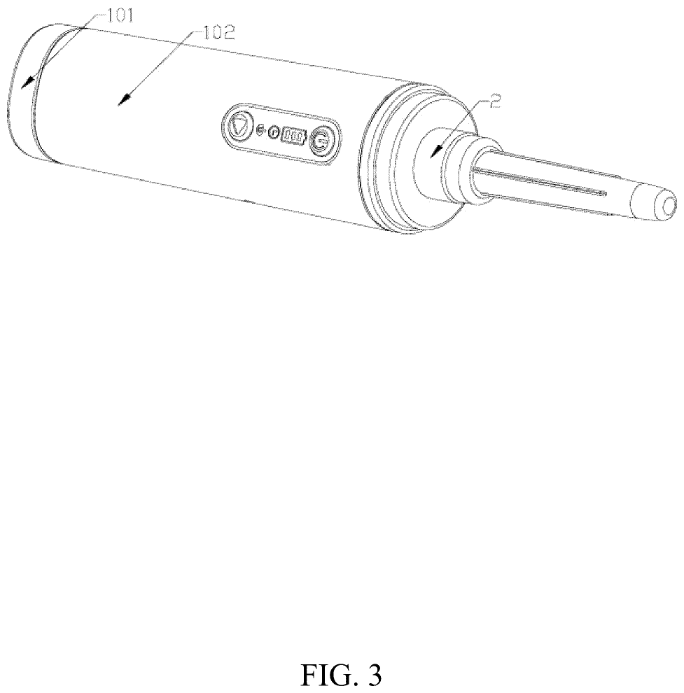

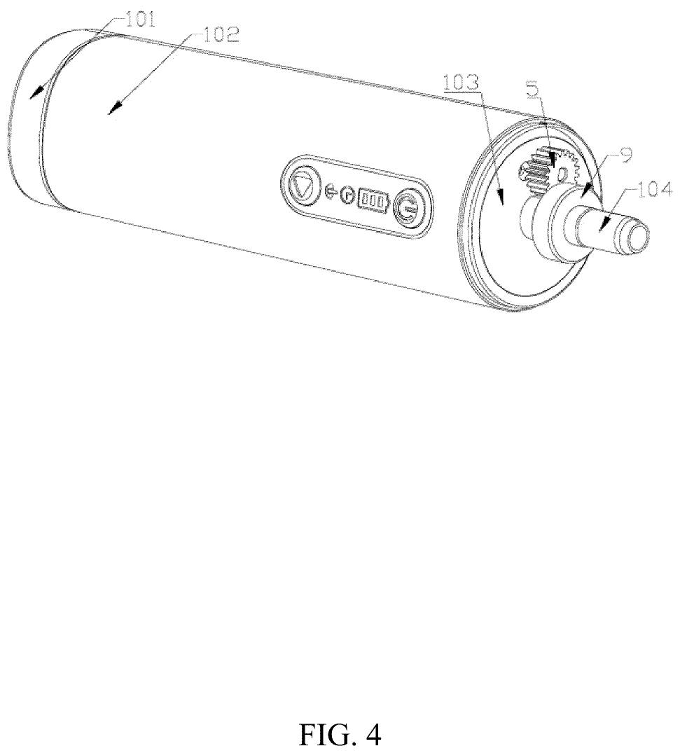

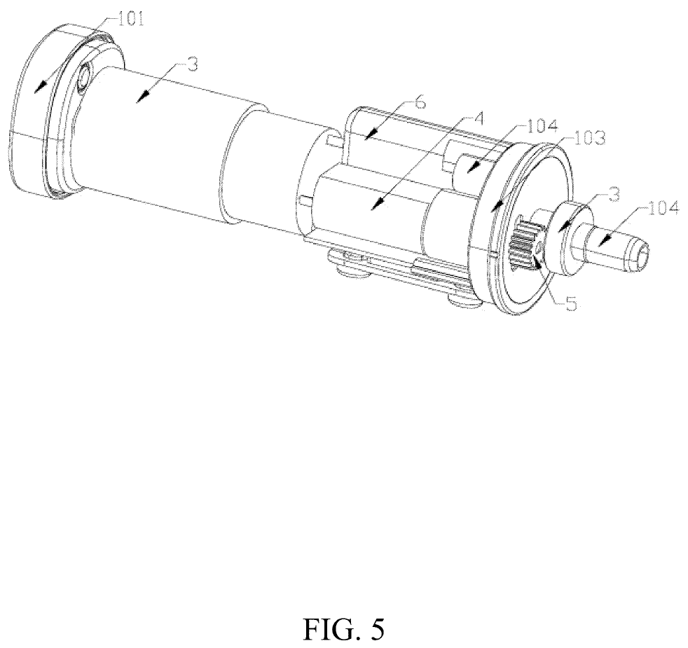

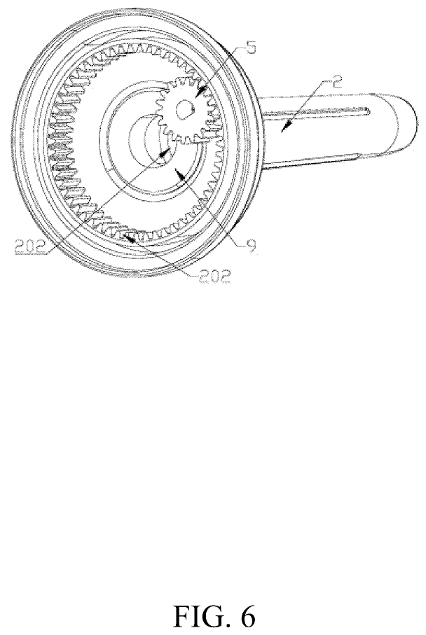

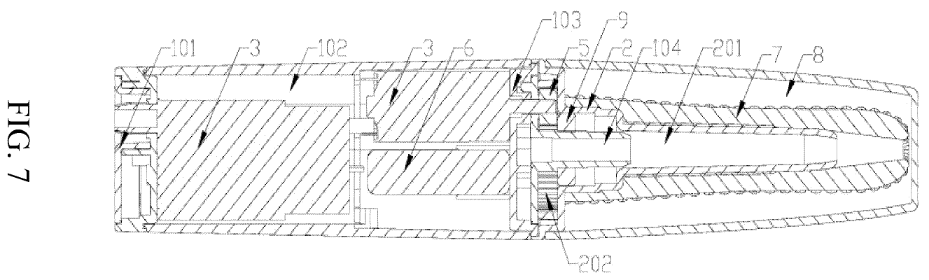

Referring to FIGS. 1-7, the present embodiment herein provides the multifunctional rotary rinser, comprising handle 1 and swivel 2; one end of the handle 1 is provided with the water inlet, which can be plugged into the hose pipe; the inlet of the hose pipe is placed in the water to be extracted to ensure the water absorption effect while placing the multifunctional rotary rinser in the desired position; the swivel 2 is rotatably connected to the other end of the handle 1; and water channel 201 is provided inside the swivel 2; the handle 1 is equipped with pump 3; and the inlet of the pump 3 is connected to the water inlet; the outlet of the pump 3 is connected to the water channel 201 through the pipe; when actual pumping is needed, the pump 3 is turned on to allow the water to pass through the pump 3 into water channel 201; and water out of water channel 201 can be used for the medical anus rinsing and daily cleaning vagina rinsing; it is also very convenient for outdoor use; the said handle 1 is also equipped with motor 4 that drives the rotation of swivel 2; the motor 4 can drive the swivel 2 to rotate, and making the water out of the channel 201 to form a rotating water spray effect, so as to achieve a better rinsing effect and the purpose of rinsing without dead corners.

The present invention herein installs pump in the handle, using the pump to connect the water inlet and water channel, and then in the use of this multifunctional rotary rinser, no longer need to rely on the role of the water heater shower, so that the outdoor and indoor use become very convenient; and at the same time, according to the need, the swivel replacement could realize the medical anus rinsing and daily cleaning vagina rinsing; the functional diversification improves the practical value; the swivel function ensures that the product will wash away the dirt inside the folds of the female vagina when flushing; when carrying out medical anal rinsing, the motor can drive the swivel to rotate, and making the water out of the channel to form a rotating water spray effect, so as to achieve a better rinsing effect and the purpose of rinsing without dead corners.

As a preferred implementation scheme in this example, it should be further explained that, the said handle 1 includes water inlet end cap 101, handle sleeve 102 and water outlet end cap 103, which are connected sequentially from one end to the other end of the handle 1; the connecting end of handle sleeve 102 can be clamped with water inlet end cap 101 and water outlet end cap 103, or it can be a threaded connection without specific restrictions, ensuring stability during use; the water inlet of the handle 1 is set on the water inlet end cap 101; the swivel 2 is rotationally connected to the water outlet cap 103; the water outlet end cap 103 is provided with the handle outlet pipe 104 connected to the water channel 201; the outlet of the pump 3 is connected to the handle outlet pipe 104, ensuring that the water flow can smoothly enter the water channel 201; the motor 4 is installed on the water outlet end cap 103 to ensure the stability of motor 4.

As a preferred implementation scheme in this example, it should be further explained that, motor shaft of the said motor (4) runs through the water outlet end cap (103) and is coaxially connected with the driving gear (5); driven gear (202) mounted in the swivel (2); driven gear (202) is meshed with driving gear (5); in this way, after the motor 4 is started, the driving gear 5 can be controlled to rotate; and then the driving gear 5 drives the driven gear 202 to rotate; the driven gear 202 then drives the entire swivel 2 to rotate, with a simple and stable structure; the driven gear 202 is fixed inside the swivel 2, so that the internal gear can be coaxially arranged with the water channel 201; the driven gear 202 can also be fitted outside the handle outlet pipe 104, without affecting the connection between the water channel 201 and the handle outlet pipe 104, making the layout of the structure more reasonable.

As a preferred implementation scheme in this example, it should be further explained that, the handle outlet pipe 104 includes inlet channel, adapter channel, and outlet channel that are sequentially connected along the direction of water flow; it can be understood that the water outlet end cap 103 is a box-shaped structure; and the adapter channel is located inside the box-shaped structure; the sealed space inside the outlet part is connected to the inlet channel and the outlet channel; in practice, even if the inlet channel and the outlet channel are not in a straight line, they can be connected through the adapter channel, making the layout of other devices simpler, and it makes the layout of other devices simpler; the end of the swivel 2 near the handle 1 is sleeved outside the water outlet end cap 103; and the middle of the swivel 2 is rotatably connected to the water outlet channel through bearing 9, ensuring the stability of swivel 2 and allowing for flexible rotation.

As a preferred implementation scheme in this example, it should be further explained that, battery 6 for supplying power to pump 3 and motor 4 is installed inside the said handle 1; charging port for charging battery 6 is provided on handle 1; the said handle 1 is also equipped with the processor; the processor is electrically connected to pump 3, motor 4, and battery 6, making control simpler.

As a preferred implementation scheme in this example, it should be further explained that, the said handle 1 is further provided with control button and power display screen, both of which are electrically connected to the processor; it is convenient and timely charging, and easier to operate.

As a preferred implementation scheme in this example, it should be further explained that, the said swivel 2 is covered with silicone case 7; water outlet is provided on the silicone case 7; furthermore, silicone case 7 can be installed as needed during use without affecting the use of the equipment; and the type and structure of silicone case 7 can be selected according to the actual situation without specific restrictions.

As a preferred technical solution in the present invention herein, protective cover 8 is provided outside the said swivel 2; when not in use, the protective cover 8 can be fastened to prevent internal components from being contaminated.

Finally, it should be noted that the above are only preferred embodiments of the present invention herein and are not intended to limit the scope of protection of the present invention herein; any modifications, equivalent substitutions, improvements, etc. made within the spirit and principles of the present invention shall be included in the scope of protection of the present invention herein.

1. Multifunctional rotary rinser, characterized in that it comprises: handle (1) and swivel (2); one end of the handle (1) is provided with the water inlet; the swivel (2) is rotationally connected to the other end of the handle (1); swivel (2) is provided with water channel (201) running through the ends of the swivel (2); pump (3) is installed in the handle (1); the inlet of the pump (3) is connected to the water inlet; the outlet of the pump (3) is connected to the water channel (201) through the pipe; the said handle (1) is also installed with motor (4) driving the swivel (2); the said handle (1) includes water inlet end cap (101), handle sleeve (102) and water outlet end cap (103), which are connected sequentially from one end to the other end of the handle (1); the water inlet of the handle (1) is set on the water inlet end cap (101); the swivel (2) is rotationally connected to the water outlet cap (103); the water outlet end cap (103) is provided with the handle outlet pipe (104) connected to the water channel (201); the outlet of the pump (3) is connected to the handle outlet pipe (104); the motor (4) is installed on the water outlet end cap (103).

2. Multifunctional rotary rinser of claim 1, characterized in that it further comprises: motor shaft of the said motor (4) runs through the water outlet end cap (103) and is coaxially connected with the driving gear (5); driven gear (202) mounted in the swivel (2); driven gear (202) is meshed with driving gear (5); the said driven gear (202) is fixed inside the swivel (2).

3. Multifunctional rotary rinser of claim 1, characterized in that it further comprises: the said handle outlet pipe (104) comprises inlet channel, adapter channel and outlet channel connected sequentially along the water flow direction; the end of the swivel (2) close to the handle (1) is socketed outside the water outlet end cap (103); the middle part of the swivel (2) is rotationally connected to the water outlet channel through bearing (9).

4. Multifunctional rotary rinser of claim 1, characterized in that it further comprises: battery (6) for supplying power to pump (3) and motor (4) is installed inside the said handle (1); charging port for charging battery (6) is provided on handle (1); the said handle (1) is also equipped with the processor; the processor is electrically connected to pump (3), motor (4), and battery (6).

5. Multifunctional rotary rinser of claim 4, characterized in that it further comprises: the said handle (1) is further provided with control button and power display screen, both of which are electrically connected to the processor.

6. Multifunctional rotary rinser of claim 1, characterized in that it further comprises: the said swivel (2) is covered with silicone case (7); water outlet is provided on the silicone case (7).

7. Multifunctional rotary rinser of claim 1, characterized in that it further comprises: protective cover (8) is provided outside the said swivel (2).
